(19) United States
(12) Patent Application Publication
(10) Pub. No.: US 2024/0270794 A1
(43) Pub. Date: Aug. 15, 2024

(54) F-18-LABELED COMPOUND FOR POSITRON EMISSION TOMOGRAPHY OF DEAD CELLS AND PREPARATION METHOD THEREFOR

(71) Applicant: FUTURECHEM CO., LTD., Seoul (KR)

(72) Inventors: Dae Yoon CHI, Seoul (KR); Keumrok OH, Seoul (KR)

(73) Assignee: FUTURECHEM CO., LTD., Seoul (KR)

(21) Appl. No.: 18/568,148

(22) PCT Filed: Jun. 10, 2022
(86) PCT No.: PCT/KR2022/008236

§ 371 Date: Jan. 12, 2024

(30) Foreign Application Priority Data
Jun. 11, 2021 (KR) 10-2021-0075817

(51) Int. Cl. C07K 7/64; A61K 51/08; C07B 59/00

(52) U.S. Cl. CPC C07K 7/64; A61K 51/08; C07B 59/008

(57) ABSTRACT

The ¹⁸F-ApoPep-7 of chemical formula 1 according to the present invention, which is a derivative of ¹⁸F-ApoPep-1, has the manufacturing advantage of labeling the radioisotope F-18 with high radiochemical yield, radiochemical purify, and high specific radioactivity. Therefore, when used as an imaging drug for positron emission tomography, the ¹⁸F-ApoPep-7 of the present invention can be applied to the diagnosis of various diseases associated with apoptosis.

### BACKGROUND OF THE INVENTION

### 1. Field of the Invention

The present invention relates to an F-18-labeled peptide compouSpnd that can be used for disease diagnosis by imaging dead cells related to various diseases and a method for producing the same.

### 2. Description of the Related Art

Apoptosis refers to a phenomenon of programmed cell death that occurs in various human tissues. Which is induced by cellular changes such as cell hemorrhage caused by biochemical substances, asymmetrical addition of cell walls, cell shrinkage, intracellular nuclear division, chromatin aggregation, and chromosomal DNA division. In the case of a normal adult, it is known that tens of billions of cells undergo apoptosis every day. However, abnormal situations where apoptosis is excessively activated or suppressed can be the cause of various diseases.

For example, in the case of tumor cells, the normal apoptotic process is suppressed and the cells continue to divide, and when an appropriate anticancer agent is administered, the suppressed apoptosis is reactivated and the tumor cells are killed. Treatment methods for tumor cells vary depending on the type, and anticancer agents must be selected according to the type of tumor cell.

In addition, when treated with anticancer drugs, cell death may appear differently for each person, so personalized medicine that measures the degree of apoptosis and adjusts the dosage and type of anticancer agents is expected to be of great help to patients.

Annexin V protein, which is currently well known as a substance for imaging the process of apoptosis in various clinical fields, was found to selectively and strongly bind to phosphatidylserine, which is abundantly distributed on the surface of apoptotic cells. However, annexin V is a 36 kD macromolecule that is disadvantageous in terms of pharmacokinetics, and has a low target signal/noise ratio because it moves slowly in vivo and takes a long time to accumulate at the target point. Therefore, annexin V, which is labeled with a positron emission radioisotope with a relatively short half-life, has limitations in human imaging through positron emission tomography.

Recently, a new peptide that specifically binds to apoptotic cells has been developed through the phage-display method and is known as ApoPep-1 with the amino acid sequence CORPPR (SEQ. ID. NO: 1), which consists of six amino acids. Unlike most existing peptides or proteins that bind to phosphatidylserine present on the surface of dead cells, ApoPep-1 has the characteristic of binding to histone H1, known as a nuclear protein, and binds to histone H1 protein exposed to the cell surface during apoptosis.

Therefore, if ApoPep-1 peptide, which specifically recognizes dead cells, is labeled with a positron emission isotope, it can be used as an imaging medicine for positron emission tomography to diagnose various diseases associated with apoptosis, including tumors.

Accordingly, the present inventors have confirmed an ¹⁸F-ApoPep-7 compound, a derivative of ¹⁸F-ApoPep-1, and an easy method for producing the same, thereby completing the present invention.

### SUMMARY OF THE INVENTION

It is an object of the present invention to provide a compound of ¹⁸F-ApoPep-7.

It is another object of the present invention to provide a compound represented by formula 5 of the present specification for producing ¹⁸F-ApoPep-7.

It is another object of the present invention to provide a method for producing ¹⁸F-ApoPep-7.

It is another object of the present invention to provide a composition for diagnosing diseases associated comprising compound represented by with apoptosis, a formula 1 of the present specification.

It is another object of the present invention to provide a PET imaging agent comprising a compound represented by formula 1 of the present specification.

To achieve the above objects, the present invention provides a compound represented by formula 1 below, a stereoisomer thereof, a solvate thereof, a hydrate thereof, or a pharmaceutically acceptable salt thereof:

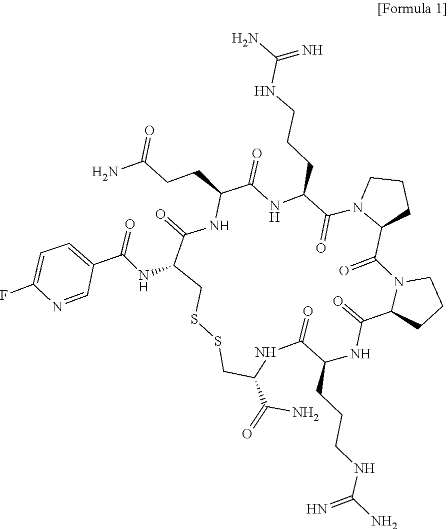 [SMILES: N=C(N)NCCC[C@@H]1NC(=O)[C@@H]2CCCN2C(=O)[C@@H]2CCCN2C(=O)[C@H](CCCNC(=N)N)NC(=O)[C@H](CCC(N)=O)NC(=O)[C@@H](NC(=O)c2ccc(F)nc2)CSSC[C@@H](C(N)=O)NC1=O]

In another aspect, the present invention provides a compound represented by formula 6 below, a stereoisomer thereof, a solvate thereof, a hydrate thereof, or a pharmaceutically acceptable salt thereof:

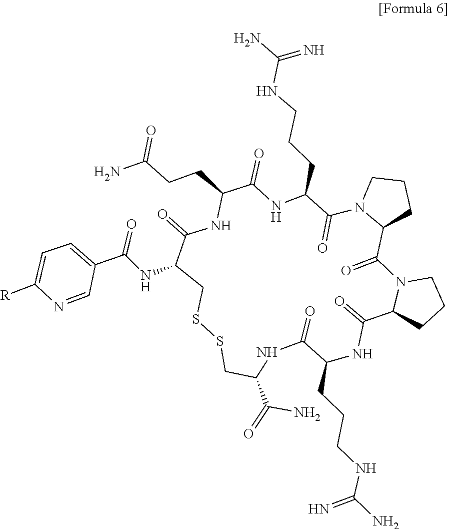

In formula 6 above, R is N⁺R¹R²R³; wherein R¹, R², and R³ are independently C1-C10 alkyl.

In another aspect, the present invention provides a method for producing a compound represented by formula 1, comprising a step of substituting a compound represented by formula 5, which is a starting material, with fluorine, as shown in reaction formula 1 below:

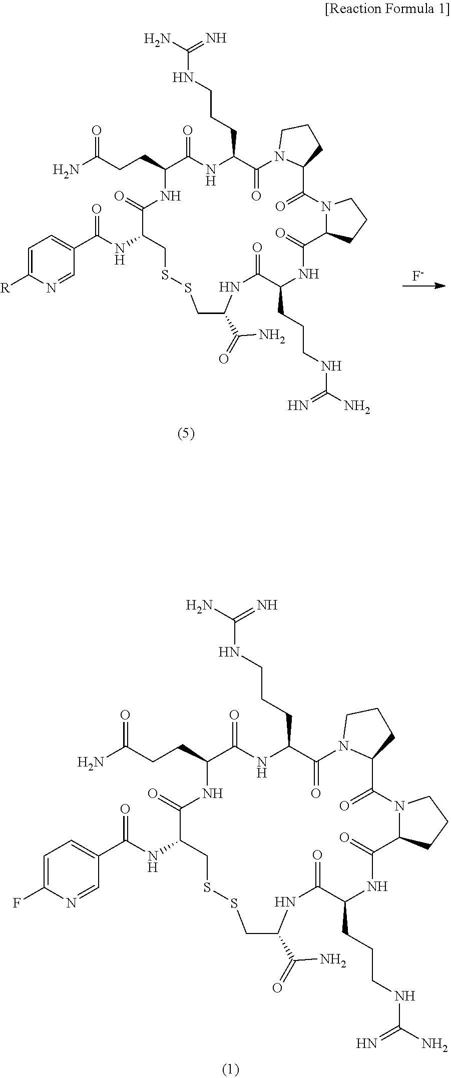

In reaction formula 1 above, F is ¹⁸F or ¹⁹F.

In another aspect, the present invention provides a composition for diagnosing diseases associated with apoptosis, comprising a compound represented by formula 1 of the present specification, a stereoisomer thereof, a solvate thereof, a hydrate thereof, or a pharmaceutically acceptable salt thereof as an active ingredient.

In another aspect, the present invention provides a PET imaging agent comprising a compound represented by formula 1 of the present specification, a stereoisomer thereof, a solvate thereof, a hydrate thereof, or a pharmaceutically acceptable salt thereof as an active ingredient.

### Advantageous Effect

The ¹⁸F-ApoPep-7 of formula 1 according to the present invention has the manufacturing advantage of labeling the radioisotope F-18 with high radiochemical yield, radiochemical purify, and high specific radioactivity. Therefore, when used as an imaging drug for positron emission tomography, the ¹⁸F-ApoPep-7 of the present invention can be applied to the diagnosis of various diseases associated with apoptosis.

### DESCRIPTION OF THE PREFERRED EMBODIMENTS

Hereinafter, the present invention is described in detail.

The embodiments of this invention can be modified in various other forms, and the scope of the present invention is not limited to the embodiments described below. It is well understood by those in the art who has the average knowledge on this field that the embodiments of the present invention are given to explain the present invention more precisely.

In addition, the “inclusion” of an element throughout the specification does not exclude other elements, but may include other elements, unless specifically stated otherwise.

In one embodiment, the present invention provides a compound represented by formula 1, a stereoisomer thereof, a solvate thereof, a hydrate thereof, or a pharmaceutically acceptable salt thereof:

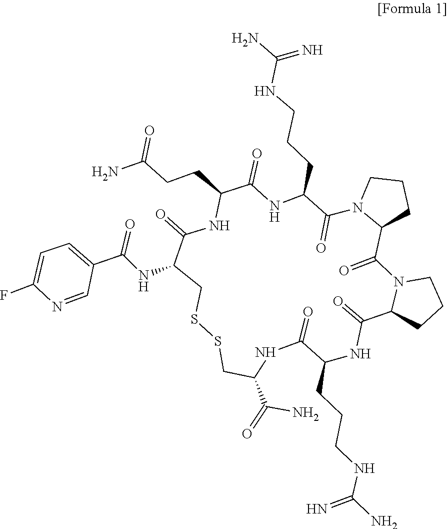 [SMILES: N=C(N)NCCC[C@@H]1NC(=O)[C@@H]2CCCN2C(=O)[C@@H]2CCCN2C(=O)[C@H](CCCNC(=N)N)NC(=O)[C@H](CCC(N)=O)NC(=O)[C@@H](NC(=O)c2ccc(F)nc2)CSSC[C@@H](C(N)=O)NC1=O]

In one embodiment, the present invention provides a compound represented by formula 1 of the present specification wherein F is ¹⁸F or ¹⁹F, a stereoisomer thereof, a solvate thereof, a hydrate thereof, or a pharmaceutically acceptable salt thereof.

In one embodiment, the present invention provides a compound represented by formula 1 of the present specification wherein F is ¹⁸F, a stereoisomer thereof, a solvate thereof, a hydrate thereof, or a pharmaceutically acceptable salt thereof.

In one embodiment, the present invention provides a compound represented by formula 6 below, a stereoisomer thereof, a solvate thereof, a hydrate thereof, or a pharmaceutically acceptable salt thereof:

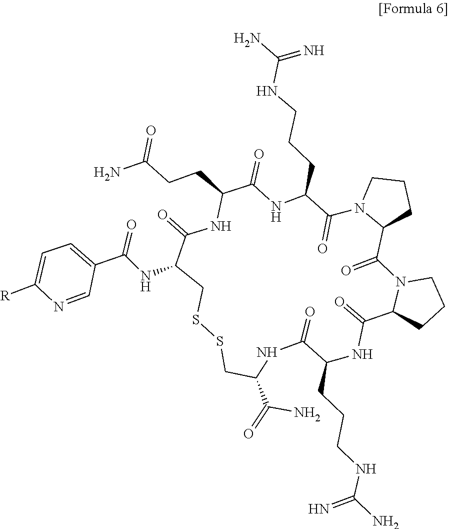

In formula 6 above, R is N⁺R¹R²R³; wherein R¹, R², and R³ are independently C1-C10 alkyl.

The compound of Formula 6 may form a salt with an anion, wherein the anion may be sulfonate, and the sulfonate may be triflate, mesylate, or tosylate.

In one embodiment, the present invention provides a compound represented by formula 6 of the present specification, wherein R¹, R², and R³ are independently C1-C5 alkyl, a stereoisomer thereof, a solvate thereof, a hydrate thereof, or a pharmaceutically acceptable salt thereof.

In one embodiment, the present invention provides a compound represented by formula 6 of the present specification, wherein R¹, R², and R³ are methyl, a stereoisomer thereof, a solvate thereof, a hydrate thereof, or a pharmaceutically acceptable salt thereof.

In one embodiment, the present invention provides a method for producing a compound represented by formula 1, comprising a step of substituting a compound represented by formula 5, which is a starting material, with fluorine (step 1), as shown in reaction formula 1 below:

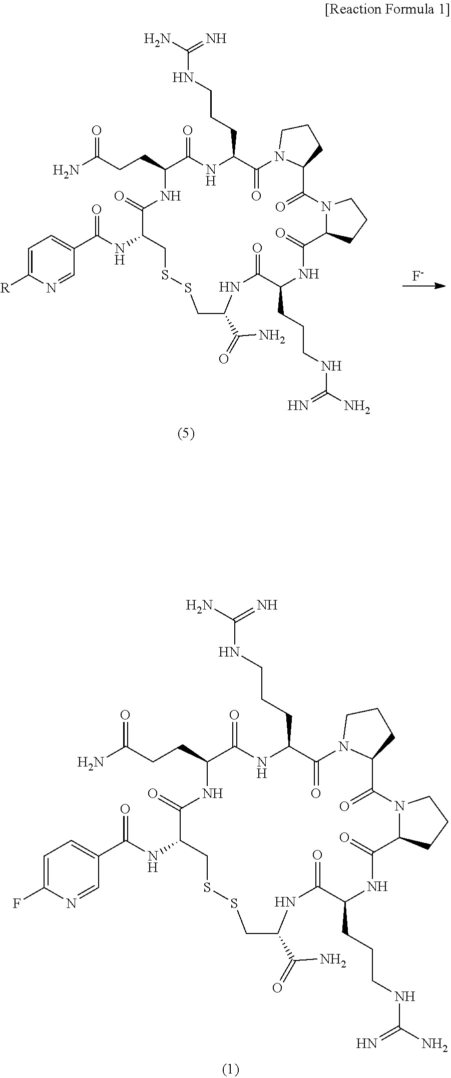

In reaction formula 1 above, F is ¹⁸F or ¹⁹F.

The reaction condition temperature may be 0° C.-100, preferably 40-60. The solvent can include common alcohols (t-butyl alcohol, amyl alcohol, etc.), acetonitrile, tetrahydrofuran, ethers (dimethyl etherdiethyl ether, etc.), dimethylformamide (DMF), and dimethylsulfoxide (DMSO).

In one embodiment, the present invention provides a imaging agent comprising a compound represented by PET formula 1 of the present invention, a stereoisomer thereof, a solvate thereof, a hydrate thereof, or a pharmaceutically acceptable salt thereof as an active ingredient.

In one embodiment, the present invention provides a composition for diagnosing diseases associated with apoptosis, comprising a compound represented by formula 1 of the present invention, a stereoisomer thereof, a solvate thereof, a hydrate thereof, or a pharmaceutically acceptable salt thereof as an active ingredient.

The composition can bind to phosphatidylserine on the surface of dead cells.

In addition, the composition can be used for positron emission tomography of apoptotic cells. The diagnostic composition can be used to image apoptosis of cancer cells after administration of an anticancer agent.

The cancer can be selected from the group consisting of pseudomyxoma, intrahepatic biliary tract cancer, hepatoblastoma, liver cancer, thyroid cancer, medullary thyroid cancer, colon cancer, testicular cancer, myelodysplastic syndrome, glioblastoma, oral cancer, lip cancer, mycosis fungoides, acute myeloid leukemia, acute lymphocytic leukemia, basal cell carcinoma, ovarian epithelial cancer, ovarian germ cell cancer, male breast cancer, brain cancer, pituitary adenoma, multiple myeloma, gallbladder cancer, biliary tract cancer, colorectal cancer, chronic myeloid leukemia, chronic lymphocytic leukemia, retinoblastoma, choroidal melanoma, ampulla of vater cancer, bladder cancer, peritoneal cancer, parathyroid cancer, adrenal cancer, nasal cavity cancer, non-small cell lung cancer, tongue cancer, astrocytoma, small cell lung cancer, pediatric brain cancer, pediatric lymphoma, pediatric leukemia, small bowel cancer, meningioma, esophageal cancer, glioma, renal pelvic cancer, kidney cancer, heart cancer, duodenal cancer, malignant soft tissue cancer, malignant bone cancer, malignant lymphoma, malignant mesothelioma, malignant melanoma, eye cancer, vulvar cancer, ureteral cancer, urethral cancer, cancer of unknown primary site, gastric lymphoma, stomach cancer, gastric carcinoid, gastrointestinal stromal cancer, Wilms cancer, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid, vaginal cancer, spinal cord cancer, acoustic tumor, pancreatic cancer, salivary gland cancer, Kaposi's sarcoma, Paget's disease, tonsil cancer, squamous cell carcinoma, lung adenocarcinoma, lung cancer, lung squamous cell carcinoma, skin cancer, anal cancer, rhabdomyosarcoma, laryngeal cancer, pleural cancer, blood cancer, and thymic cancer.

Further, the composition can be used in the diagnosis of neurodegenerative diseases.

The neurodegenerative disease can be selected from the group consisting of Alzheimer's disease, Parkinson disease, Wilson disease, neurodegeneration due to traumatic brain injury, neurodegeneration due to spinal cord injury, neurodegeneration due to stroke, amyotrophic lateral sclerosis, human immunodeficiency viral dementia, Huntington's disease, multiple sclerosis, cerebral amyloid angiopathy, and tauopathy.

The said neurodegenerative disease targets nerve cells damaged by the accumulation of beta-amyloid and increased apoptosis.

The disease associated with apoptosis of the present invention can be selected from the group consisting of stroke, angina pectoris, and myocardial infarction.

The positron emission tomography of the present invention is capable of imaging apoptosis in response to anticancer treatment, allowing confirmation of the effect of apoptosis within a short time.

The composition of the present invention can be used in the diagnosis of f inflammatory and in the inflammatory tissue, macrophages process apoptotic cells by ingesting them.

The composition of the present invention can be used in the diagnosis of autoimmune disease in which apoptosis is actively occurring, such as rejection after organ transplantation.

Hereinafter, the present invention will be described detail by the following examples. However, the in following examples are only for illustrating the present invention, and the contents of the present invention are not limited thereto.

### Example 1: Preparation of linear-ApoPep-1-cys-amide Compound (2)

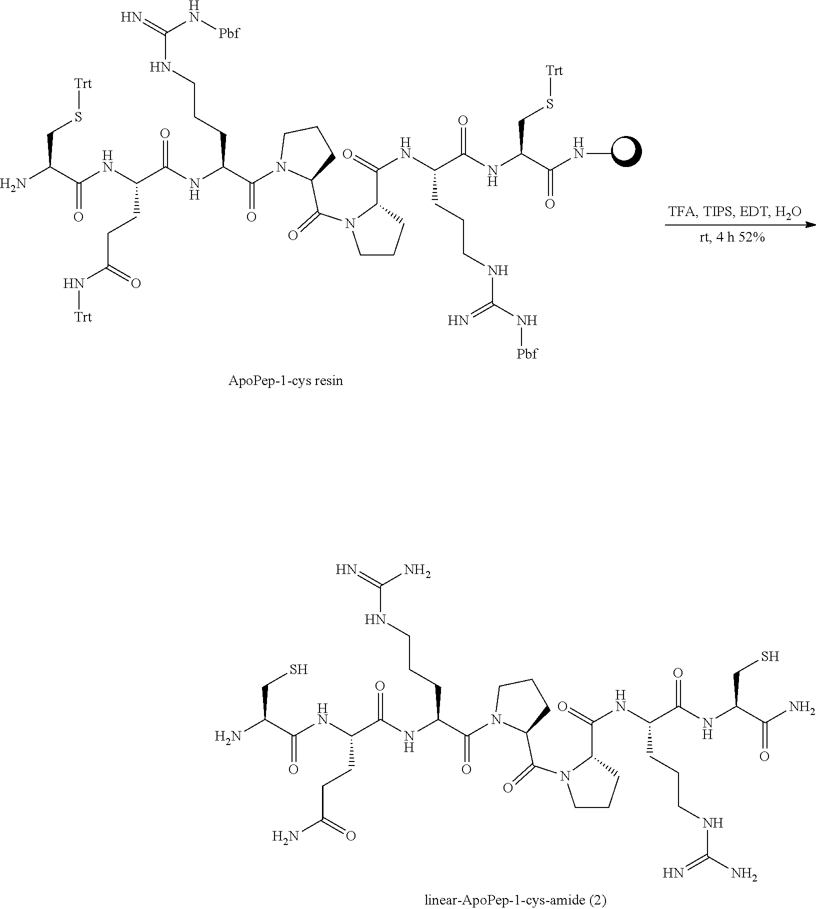

A peptide (ApoPep-1-cys resin) with the amino acid sequence Cys-Gln-Arg-Pro-Pro-Arg-Cys protected by a protecting group and linked to a polymer by a protecting group was prepared according to a general amino acid synthesis method. ApoPep-1-cys resin was separated from resin by using a solution (trifluoroacetic acid (TFA):triisopropylsilane (TIPS):1,2-ethanediol (EDT):water=94:2:2:2) for 4 hours at room temperature. The resin was washed with 15 mL of the same solution as above and then washed with water. Diethyl ether was added to the solution to obtain a solid. The resulting solid was dissolved in 3 mL of water, and this solution was purified by reverse-phase high-performance liquid chromatography (RP-HPLC) to obtain compound 2 (0.61 g, 52%) as a white powder. The compound 2 has a modified amino acid sequence CORPPRC-NH₂ (SEQ. ID. NO: 2) in which the cysteine linked to the C-terminus of ApoPep-1 is amidated.

HRMS (ESI⁺): m/z calcd. for C₃₃H₆₀N₁₅O₈S₂⁺ [M+H]⁺: 858.4185, found 858.4186

### Example 2: Preparation of cyclic-ApoPep-1-cys-amide Compound (3)

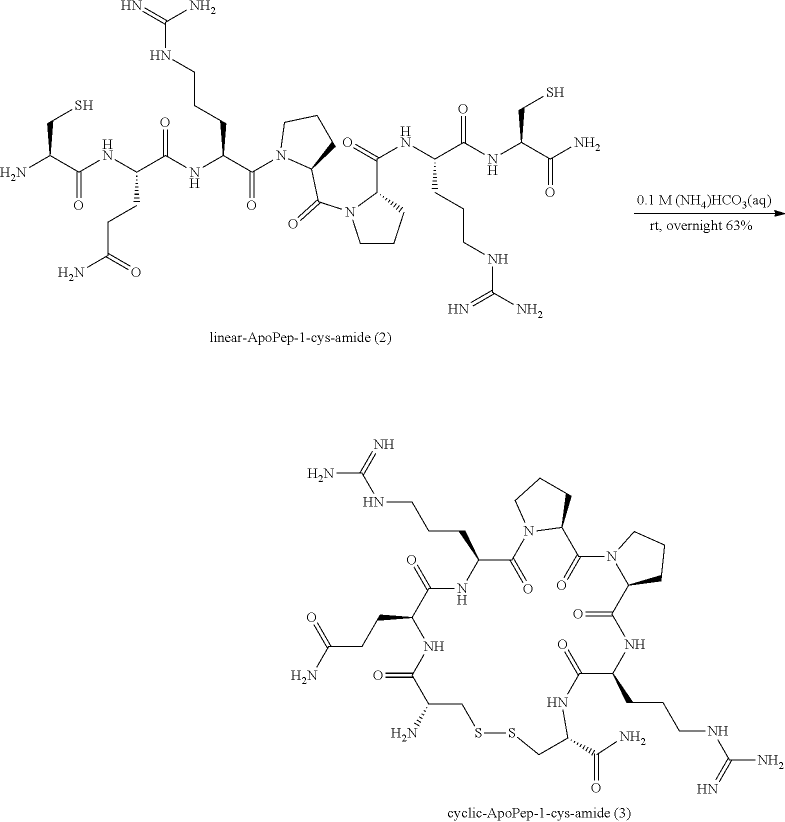 [SMILES: N=C(N)NCCC[C@H](NC(=O)[C@@H]1CCCN1C(=O)[C@@H]1CCCN1C(=O)[C@H](CCCNC(=N)N)NC(=O)[C@H](CCC(N)=O)NC(=O)[C@@H](N)CS)C(=O)N[C@@H](CS)C(N)=O ; N=C(N)NCCC[C@@H]1NC(=O)[C@@H]2CCCN2C(=O)[C@@H]2CCCN2C(=O)[C@H](CCCNC(=N)N)NC(=O)[C@H](CCC(N)=O)NC(=O)[C@@H](N)CSSC[C@@H](C(N)=O)NC1=O]

The compound 2 (610 mg, 0.71 mmol) prepared in Example 1 was dissolved in 0.1 M ammonium hydrogen carbonate solution (100 mL), and then stirred at room temperature overnight. The resulting mixture was concentrated in vacuo. The residue was dissolved in HPLC grade water (3 mL) and purified by reverse phase high performance liquid chromatography (RP-HPLC) to give compound 3 (383 mg, 63%) as a white powder. The compound 3 is an amino acid sequence in which the linear amino acid sequence of SEQ. ID. NO: 2 is cyclized through a disulfide bond.

HRMS (ESI+): m/z calcd. for C33H58N15O8S2+ [M+H]+; 856.4029, found 856.4029.

### Example 3: Preparation of Compound 5

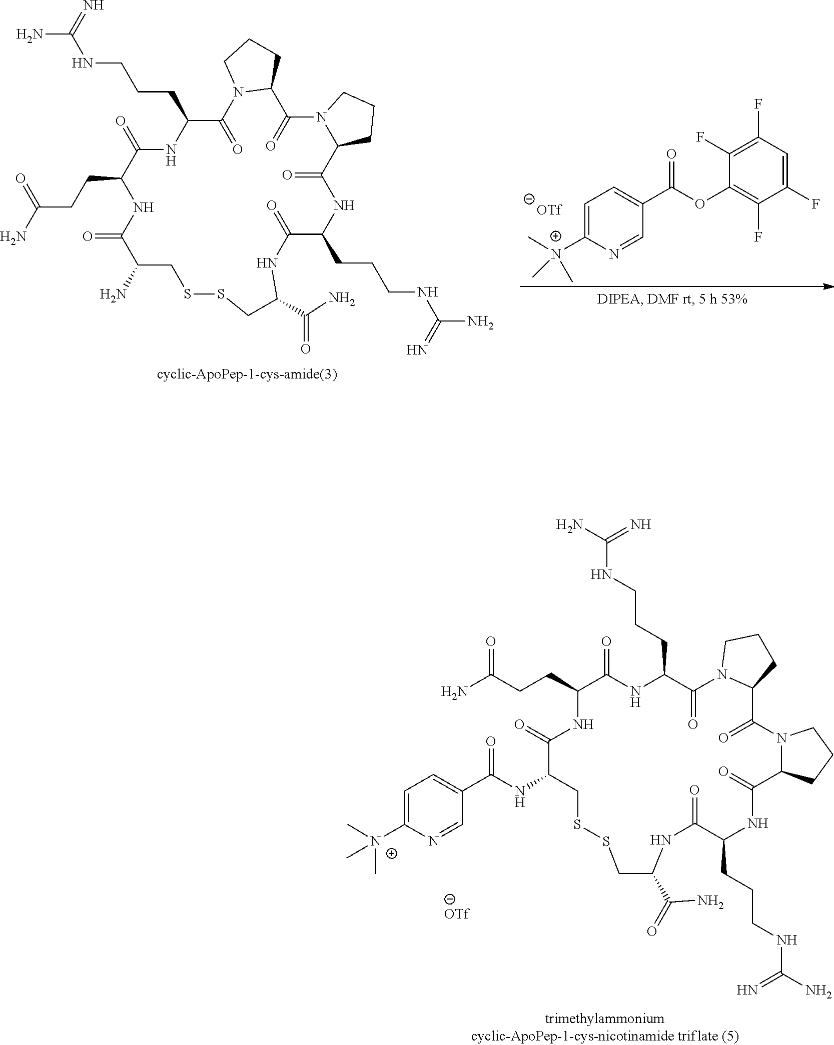 [SMILES: N=C(N)NCCC[C@@H]1NC(=O)[C@@H]2CCCN2C(=O)[C@@H]2CCCN2C(=O)[C@H](CCCNC(=N)N)NC(=O)[C@H](CCC(N)=O)NC(=O)[C@@H](N)CSSC[C@@H](C(N)=O)NC1=O ; C[N+](C)(C)c1ccc(C(=O)Oc2c(F)c(F)cc(F)c2F)cn1 ; C[N+](C)(C)c1ccc(C(=O)N[C@H]2CSSC[C@@H](C(N)=O)NC(=O)[C@H](CCCNC(=N)N)NC(=O)[C@@H]3CCCN3C(=O)[C@@H]3CCCN3C(=O)[C@H](CCCNC(=N)N)NC(=O)[C@H](CCC(N)=O)NC2=O)cn1]

The compound 3 produced in Example 2 and compound 4 were prepared. Compound 3 (102.7 mg, 0.12 mmol) was added to a solution in which compound 4 (61.5 mg, 0.10 mmol) and N,N-diisopropylethylamine (52.3 μl, 0.30 mmol) were dissolved in DMF (2 mL). The reactant was stirred at room temperature for 5 hours. The residue was dissolved in deionized water (3 mL), and this solution was purified by reverse phase high performance liquid chromatography (RP-HPLC) to give compound 5 (82.7 mg, 53%) as a white powder.

HRMS (ESI⁺): m/z calcd. for C₁₅H₁₃F₄N₂O₂⁺ [M-CF₃SO₃]⁺: 329.0908, found: 329.0908.

Compound 4 was prepared according to a known method. Specifically, it was prepared reference to the with following literature (Ryan A. Davis, et al., Acta Crystallogr C Struct Chem. 2018).

N,N,N-Trimethyl-5-((2,3,5,6-tetrafluorophenoxy) carbonyl)pyridin-2-aminium trifluoromethanesulfonate (4)

¹H NMR (400 MHZ, CD₃CN) δ 9.33 (s, 1H), 8.85 (d, J=8.7 Hz, 1H), 8.10 (d, J=8.7 Hz, 1H), 7.49-7.37 (m, 1H), 3.61 (s, 9H).

¹³C NMR (100 MHZ, CD₃SOCD₃) δ 164.8, 159.1, 149.3, 147.6-147.3 (m), 144.4-144.0 (m), 141.8, 139.6-139.3 (m), 136.9-136.5 (m), 136.3-136.1 (m), 128.9, 120.7 (q, J=322.5 Hz), 115.6, 95.4 (t, J=23.9 Hz), 54.6.

### Example 4: Preparation of ApoPep-7 Compound 1

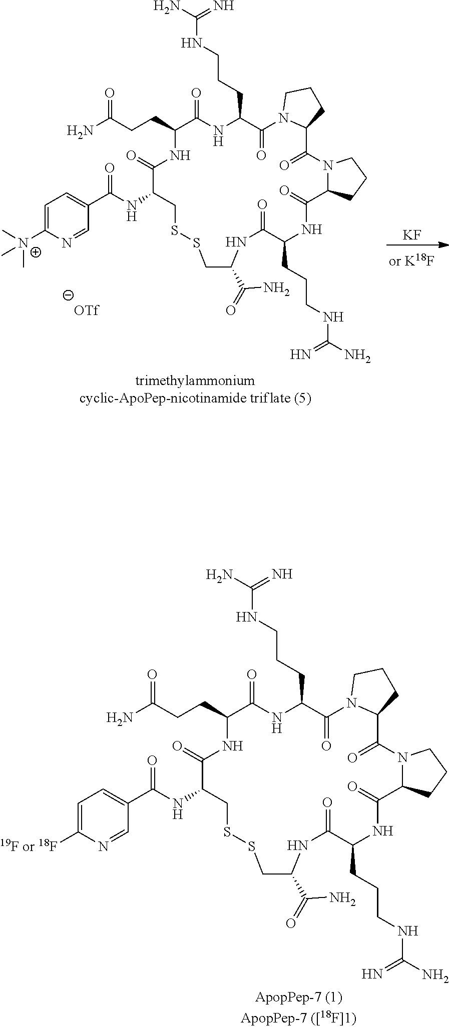

Prior to the substitution reaction of ¹⁸F, the substitution reaction of non-radioactive fluoride was performed.

Compound 5 (35 mg, 0.03 mmol), the precursor, was dissolved in acetonitrile (4 mL), to which Kryptofix222/KF complex (19.6 mg, 0.045 mmol) was added at room temperature for 10 minutes. Then, the reactant was concentrated in vacuo, dissolved in H₂O/CH₃CN (0.8 mL/0.6 mL), and purified by RP-HPLC to give compound 1 (27 mg, 93%) as a white powder. HPLC purification was performed with an Altima HP C18 5μ 250×10 mm semi-prep column eluting at a rate of 4 mL/minute using a solvent gradient (0-100% CH3CN/0.1% TFA in H2O for 20 min).

HRMS (ESI⁺): m/z calcd. for C39H60FN16O9S2+[M+H]+: 979.4149, found: 979.4149.

Subsequently, ¹⁸F substitution reaction was performed.

¹⁸F (432.9 MBq) was passed through an anion exchange resin (QMA). Using a solution of Kryptofix222/KOMs complex (10 mg) dissolved in ethanol (1 mL), ¹⁸F was eluted into a reaction vial. The solution was concentrated to dryness by heating at 100° C. under nitrogen. The compound 5 (4.0 mg, 3.9 μmol) dissolved in 0.5 mL of acetonitrile was added to the dried Kryptofix222/K18F complex. The solution was heated at 40° C. for 15 minutes and then analyzed by radio-TLC (80%). Compound [¹⁸F]1 (31%, non-decay-corrected) was isolated by purification with reverse-phase high-performance liquid chromatography (RP-HPLC).

Meanwhile, the radiochemical yield for the fluorine substitution reaction of the compound represented by formula 5 was confirmed under various conditions, and the results are shown in table 1 below.

Table 1 shows the yields confirmed by radio thin layer chromatography (radio-TLC) after fluorinated labeling (TLC RCY) and the yields after purification (RCY).

TABLE 1

| | Precursor | | | | Reaction | TLC | |
|---|---|---|---|---|---|---|---|
| No | amount | | Activity | Temp | time | RCY | RCY |
| 1 | 3.8 | mg | 66.6 | 40° C. | 30 min | 54% | 17% (11.1 Mbq) |
| | | | Mbq | | | | for 75 min |
| 2 | 4.0 | mg | 432.9 | 40° C. | 15 min | 80% | 31% (133.2 Mbq) |
| | | | Mbq | | | | for 65 min |
| 3 | 4.1 | mg | 225.7 | 42° C. | 15 min | 94% | 30% (70.3 Mbq) |
| | | | Mbq | | | | for 55 min |
| 4 | 3.7 | mg | 1150.7 | 40° C. | 15 min | 82% | 26% (307.1 Mbq) |
| | | | Mbq | | | | for 65 min |
| 5 | 4.2 | mg | 11.2 | 60° C. | 15 min | 92% | 31% (3.4 Gbq) |
| | | | Gbq | | | | for 65 min |

What is claimed is:

1. A compound represented by formula 1 below, a stereoisomer thereof, a solvate thereof, a hydrate thereof, or a pharmaceutically acceptable salt thereof:

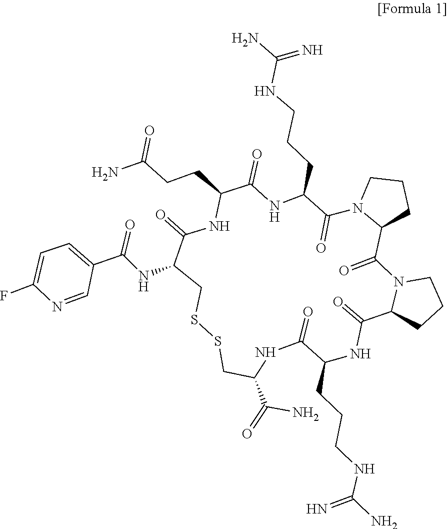 [SMILES: N=C(N)NCCC[C@@H]1NC(=O)[C@@H]2CCCN2C(=O)[C@@H]2CCCN2C(=O)[C@H](CCCNC(=N)N)NC(=O)[C@H](CCC(N)=O)NC(=O)[C@@H](NC(=O)c2ccc(F)nc2)CSSC[C@@H](C(N)=O)NC1=O]

2. The compound, the stereoisomer thereof, the solvate thereof, the hydrate thereof, or the pharmaceutically acceptable salt thereof according to claim 1, wherein F is ¹⁸F or ¹⁹F.

3. The compound, the stereoisomer thereof, the solvate thereof, the hydrate thereof, or the pharmaceutically acceptable salt thereof according to claim 1, wherein F is ¹⁸F.

4. A compound represented by formula 6 below, a stereoisomer thereof, a solvate thereof, a hydrate thereof, or a pharmaceutically acceptable salt thereof: 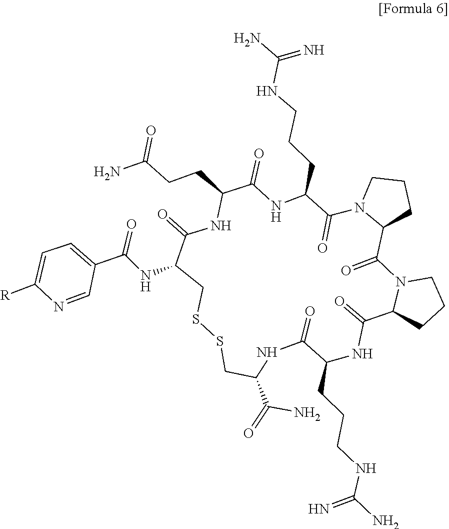
In formula 6 above, R is N⁺R¹R²R³; wherein R¹, R², and R³ are independently C1-C10 alkyl.

5. The compound, the stereoisomer thereof, the solvate thereof, the hydrate thereof, or the pharmaceutically acceptable salt thereof according to claim 4, wherein R¹, R², and R³ are independently C1-C5 alkyl.

6. The compound, the stereoisomer thereof, the solvate thereof, the hydrate thereof, or the pharmaceutically acceptable salt thereof according to claim 4, wherein R¹, R², and R³ are methyl.

7. A method for producing a compound represented by formula 1, comprising a step of substituting a compound represented by formula 5, which is a starting material, with fluorine, as shown in reaction formula 1 below: 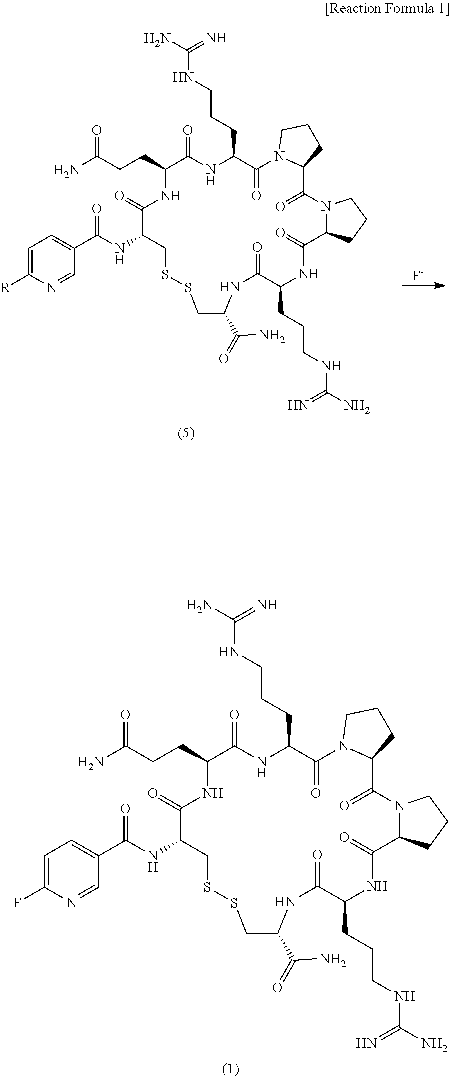
In reaction formula 1 above, F is ¹⁸F or ¹⁹F.

8. A composition for imaging apoptosis, comprising a compound represented by formula 1 of claim 1, a stereoisomer thereof, a solvate thereof, a hydrate thereof, or a pharmaceutically acceptable salt thereof as an active ingredient.

9. The composition according to claim 8, wherein the composition binds to phosphatidylserine on the surface of apoptotic cells.

10. The composition according to claim 8, wherein the composition is used for positron emission tomography of apoptotic cells.

11. The composition according to claim 8, wherein the composition is used to image apoptosis of cancer cells after administration of an anticancer agent.

12. The composition according to claim 11, wherein the cancer is at least one selected from the group consisting of pseudomyxoma, intrahepatic biliary tract cancer, hepatoblastoma, liver cancer, thyroid cancer, medullary thyroid cancer, colon cancer, testicular cancer, myelodysplastic syndrome, glioblastoma, oral cancer, lip cancer, mycosis fungoides, acute myeloid leukemia, acute lymphocytic leukemia, basal cell carcinoma, ovarian epithelial cancer, ovarian germ cell cancer, male breast cancer, brain cancer, pituitary adenoma, multiple myeloma, gallbladder cancer, biliary tract cancer, colorectal cancer, chronic myeloid leukemia, chronic lymphocytic leukemia, retinoblastoma, choroidal melanoma, ampulla of vater cancer, bladder cancer, peritoneal cancer, parathyroid cancer, adrenal cancer, nasal cavity cancer, non-small cell lung cancer, tongue cancer, astrocytoma, small cell lung cancer, pediatric brain cancer, pediatric lymphoma, pediatric leukemia, small bowel cancer, meningioma, esophageal cancer, glioma, renal pelvic cancer, kidney cancer, heart cancer, duodenal cancer, malignant soft tissue cancer, malignant bone cancer, malignant lymphoma, malignant mesothelioma, malignant melanoma, eye cancer, vulvar cancer, ureteral cancer, urethral cancer, cancer of unknown primary site, gastric lymphoma, stomach cancer, gastric carcinoid, gastrointestinal stromal cancer, Wilms cancer, breast cancer, sarcoma, penile cancer, pharyngeal cancer, gestational choriocarcinoma, cervical cancer, endometrial cancer, uterine sarcoma, prostate cancer, metastatic bone cancer, metastatic brain cancer, mediastinal cancer, rectal cancer, rectal carcinoid, vaginal cancer, spinal cord cancer, acoustic tumor, pancreatic cancer, salivary gland cancer, Kaposi's sarcoma, Paget's disease, tonsil cancer, squamous cell carcinoma, lung adenocarcinoma, lung cancer, lung squamous cell carcinoma, skin cancer, anal cancer, rhabdomyosarcoma, laryngeal cancer, pleural cancer, blood cancer, and thymic cancer.

13. The composition according to claim 8, wherein the composition is for diagnosing neurodegenerative diseases.

14. The composition according to claim 13, wherein the neurodegenerative disease is at least one selected from the group consisting of Alzheimer's disease, Parkinson disease, Wilson disease, neurodegeneration due to traumatic brain injury, neurodegeneration due to spinal cord injury, neurodegeneration due to stroke, amyotrophic lateral sclerosis, human immunodeficiency viral dementia, Huntington's disease, multiple sclerosis, cerebral amyloid angiopathy, and tauopathy.

15. The composition according to claim 8, wherein the composition is for imaging apoptosis in high-risk atherosclerotic plaque tissue.

16. The composition according to claim 8, wherein the composition is for diagnosing a disease selected from the group consisting of stroke, angina pectoris, and myocardial infarction.

17. The composition according to claim 8, wherein the composition is for imaging inflammatory tissue resulting from apoptotic processing by macrophages.

18. The composition according to claim 8, wherein the composition is for diagnosing inflammatory diseases.

19. The composition according to claim 8, wherein the composition is for diagnosing autoimmune diseases.

20. A PET imaging agent comprising a compound represented by formula 1 of claim 1, a stereoisomer thereof, a solvate thereof, a hydrate thereof, or a pharmaceutically acceptable salt thereof as an active ingredient.
